(19) United States
(12) Patent Application Publication
(10) Pub. No.: US 2022/0222607 A1
(43) Pub. Date: Jul. 14, 2022

(54) WORK SYSTEM, CONTROL METHOD, AND COMPUTER-READABLE STORAGE MEDIUM STORING PROGRAM

(71) Applicant: HONDA MOTOR CO., LTD., Tokyo (JP)

(72) Inventors: Misago ITO, Tokyo (JP); Takayuki KAWAI, Wako-shi (JP); Tsukasa SUGINO, Wako-shi (JP); Makoto YAMAMURA, Wako-shi (JP); Masaya HONJI, Wako-shi (JP)

(21) Appl. No.: 17/570,688

(22) Filed: Jan. 7, 2022

(30) Foreign Application Priority Data
Jan. 14, 2021 (JP) 2021-004486

(51) Int. Cl. G06Q 10/06; A61B 5/0205; G01W 1/10

(52) U.S. Cl. CPC G06Q 10/063114; G01W 1/10; A61B 5/02055; G06Q 10/063118

(57) ABSTRACT

A work system comprises: an acquisition unit configured to acquire biological information of a user; a creation unit configured to create a work plan in a work area; a control unit configured to control a work device for performing work in the work area on the basis of the work plan created by the creation unit; and a change unit configured to change the work plan created by the creation unit on the basis of the biological information acquired by the acquisition unit. The control unit controls the work device on the basis of the changed work plan in a case where the work plan created by the creation unit is changed by the change unit.

### CROSS-REFERENCE TO RELATED APPLICATION(S)

This application claims priority to and the benefit of Japanese Patent Application No. 2021-004486 filed on Jan. 14, 2021, the entire disclosure of which is incorporated herein by reference.

### BACKGROUND OF THE INVENTION

### Field of the Invention

The present invention relates to a work system that autonomously performs work, a control method, and a computer-readable storage medium storing a program.

### Description of the Related Art

International Publication No. 2018/135988 describes an automatic lawn mower that detects the presence of a living body and changes a work plan. Meanwhile, US-2018-0330306 describes a wearable device that detects movement of an employee and generates RAW data of daily work activities.

### SUMMARY OF THE INVENTION

It is assumed that automatic lawn mowers become widespread, and the number of scenes where users (workers) and automatic lawn mowers work in cooperation increases. In such a tendency, it is necessary to appropriately decide the work plan while considering the state of the user.

The present invention provides a work system, a control method, and a computer-readable storage medium storing a program for appropriately deciding a work plan on the basis of the state of the user.

The present invention in its first aspect provides a work system including: an acquisition unit configured to acquire biological information of a user; a creation unit configured to create a work plan in a work area; a control unit configured to control a work device for performing work in the work area on the basis of the work plan created by the creation unit; and a change unit configured to change the work plan created by the creation unit on the basis of the biological information acquired by the acquisition unit, in which the control unit controls the work device on the basis of the changed work plan in a case where the work plan created by the creation unit is changed by the change unit.

The present invention in its second aspect provides a control method executed in a work system, the control method including: acquiring biological information of a user; creating a work plan in a work area; controlling a work device for performing work in the work area on the basis of the created work plan; and changing the created work plan on the basis of the acquired biological information, in which the work device is controlled on the basis of the changed work plan in a case where the created work plan is changed.

The present invention in its third aspect provides a non-transitory computer-readable storage medium storing a program for causing a computer to function to: acquire biological information of a user; create a work plan in a work area; control a work device for performing work in the work area on the basis of the created work plan; and change the created work plan on the basis of the acquired biological information, in which the work device is controlled on the basis of the changed work plan in a case where the created work plan is changed.

It is possible with the present invention to appropriately decide the work plan on the basis of the state of the user.

### BRIEF DESCRIPTION OF THE DRAWINGS

FIG. 1 is a diagram illustrating the configuration of an automatic work system;

FIG. 2 is a diagram illustrating the configuration of an automatic work machine;

FIG. 3 is a diagram illustrating the configuration of a server;

FIG. 4 is a diagram illustrating the configuration of a wearable device;

FIG. 5 is a diagram illustrating the configuration of a mobile terminal;

FIG. 6 is a sequence diagram for explaining the operation of an automatic work system;

FIG. 7 is a flowchart illustrating processing of an automatic work machine;

FIG. 8 is a flowchart illustrating processing of creating a work plan;

FIG. 9 is a flowchart illustrating processing of changing a work plan;

FIG. 10 is a flowchart illustrating processing of a server;

FIG. 11 is a flowchart illustrating processing of adjusting a reward amount;

FIG. 12 is a diagram illustrating a state in which an area wire and a charging station are laid;

FIGS. 13A and 13B are diagrams each illustrating a user interface screen; and

FIG. 14 is a flowchart illustrating emergency processing.

### DESCRIPTION OF THE EMBODIMENTS

Hereinafter, embodiments will be described in detail with reference to the attached drawings. Note that the following embodiments are not intended to limit the scope of the claimed invention, and limitation is not made an invention that requires all combinations of features described in the embodiments. Two or more of the multiple features described in the embodiments may be combined as appropriate. Furthermore, the same reference numerals are given to the same or similar configurations, and redundant description thereof is omitted.

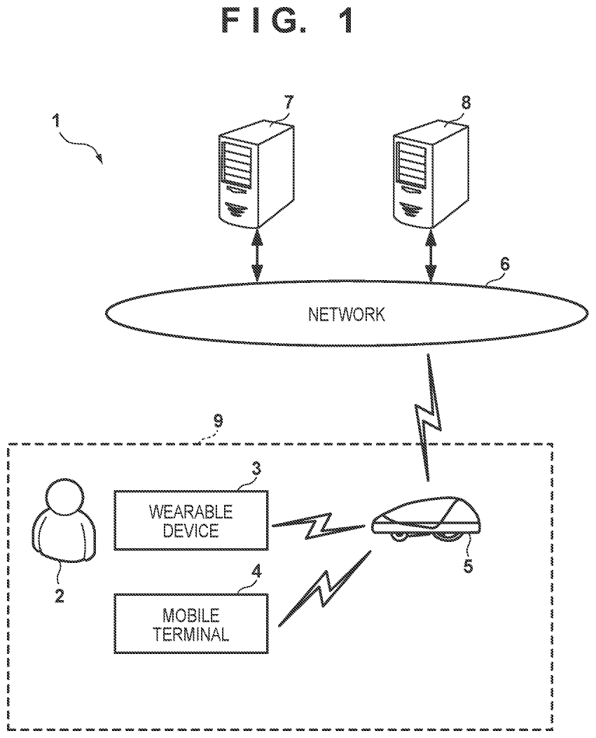

FIG. 1 is a diagram illustrating an example of the configuration of an automatic work system according to the present embodiment. An automatic work system 1 includes a wearable device 3 worn by a user 2 who is a worker, a mobile terminal 4 corresponding to the user 2, an automatic work machine 5 that performs work in a work area 9, a server 7, and a server 8. In the present embodiment, the work area 9 is an area where, for example, mowing work is performed, and the automatic work machine 5 is an automatic lawn mower that performs work while autonomously traveling in the work area 9. Although a lawn mower will be described as the automatic work machine 5 in the present embodiment, the automatic work machine may be a work machine that autonomously performs another type of work, and may be, for example, a snow blower or a cultivator. The automatic work machine 5 is activated by an operation of the user 2, and autonomously performs work in the work area 9 in accordance with a work plan. Although only one automatic work machine 5 is illustrated in FIG. 1, a plurality of automatic work machines 5 may be provided.

The wearable device 3 is worn by the user 2 so as to be able to measure biological information of the user 2. The biological information is, for example, information regarding at least one of a heart rate, a pulse, a blood pressure, a body temperature, or a respiratory amount of the user 2. The mobile terminal 4 is an information processing device corresponding to the user 2, and is, for example, a smartphone held by the user 2. In the present embodiment, the wearable device 3 and the automatic work machine 5 are communicably connected with each other, the mobile terminal 4 and the automatic work machine 5 are communicably connected with each other, and the biological information is directly transmitted from the wearable device 3 to the automatic work machine 5. However, the biological information may be transmitted to the automatic work machine 5 via the mobile terminal 4. Moreover, the wearable device 3 may be connected with a network 6, and the biological information may be transmitted to the automatic work machine 5 via a device on the network 6. The wearable device 3 and the mobile terminal 4 can communicate with the server 7 and the server 8 via the automatic work machine 5. Moreover, the wearable device 3 and the mobile terminal 4 may be connected with the network 6 so as to be able to communicate with the server 7 and the server 8.

The server 7 is a server that provides a service of the automatic work system 1, and the server 7 and the automatic work machine 5 can communicate with each other via the network 6 such as the Internet. The service provided by the server 7 is a service for which the work plan is appropriately decided on the basis of the state of the user 2, and is, for example, a service for which the work plan can be flexibly updated in accordance with the physical condition or the like of the user 2 during the mowing work. The automatic work system 1 allows the user 2 not only to cause the automatic work machine 5 to autonomously perform the lawn mowing work, but also to achieve cooperative work by causing the automatic work machine 5 to autonomously perform the lawn mowing work and performing the work by himself/herself. As an example of such cooperative work, there is a case where the user 2 himself/herself performs work at a place where work by the automatic work machine 5 is difficult, for example, in the vicinity of an end portion or an edge of the work area 9. As another example of cooperative work, there is a case where the automatic work machine 5 performs mowing work and the user 2 performs blower work.

In a case where the work area 9 is outdoors as an example of a case to which the present embodiment can be applied, it is assumed that a load applied to the user 2 increases as the temperature increases. In the present embodiment, in such a case, the automatic work machine 5 can monitor the biological information of the user 2 during the mowing work, and can flexibly change the work plan of the user 2 and the work plan of the automatic work machine 5 on the basis of the monitoring result.

The server 8 is a server for obtaining environment information of the work area 9 where the user 2 is present or the vicinity thereof. The environment information is, for example, weather information such as weather, temperature, or humidity, or information such as noise. Moreover, if there is a camera or the like in the vicinity of the work area 9, the captured image or the like is also included in the environment information. The server 7 and the server 8 can communicate with each other via the network 6, and for example, the server 7 can acquire environment information of the work area 9 or the vicinity thereof from the server 8 at an arbitrary timing, for example, at a timing when the automatic work machine 5 starts the work.

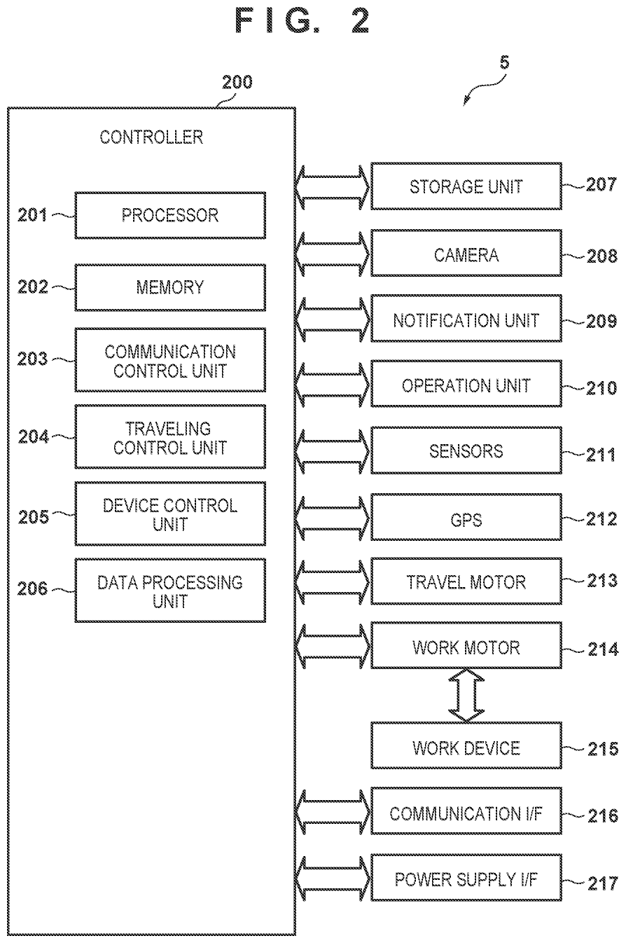

FIG. 2 is a diagram illustrating an example of the configuration of the automatic work machine 5. A controller 200 is configured as, for example, an electronic control unit (ECU), and integrally controls the automatic work machine 5. The controller 200 includes a processor 201 such as a CPU, a memory 202 such as a ROM, an EEPROM, or a RAM, a communication control unit 203, a traveling control unit 204, a device control unit 205, and a data processing unit 206. The operation of the automatic work machine 5 in the present embodiment is implemented by, for example, the processor 201 reading and executing a program stored in the memory 202. That is, a device including the controller 200 can be a computer in the present invention.

The memory 202 stores a control program and data for controlling the operation of each unit of the automatic work machine 5. For example, the memory 202 stores a traveling control program and data for speed control and position control, and a communication control program and data for communication control with the outside. The memory 202 also stores a device control program and data (height and orientation of blade, rotation speed, etc.) for controlling a work device that performs work. The memory 202 also stores a setup wizard program for setting a work area and a work plan. The automatic work machine 5 can autonomously travel and perform work on the basis of the set work plan. Moreover, the above program and data may be stored in a storage unit 207 configured outside the controller 200.

The communication control unit 203 controls communication with the outside on the basis of the communication control program and data stored in the memory 202. The traveling control unit 204 controls traveling (including a turning operation) in the work area 9 on the basis of the traveling control program and data stored in the memory 202. The device control unit 205 controls the work device 215 on the basis of the device control program and data stored in the memory 202. The data processing unit 206 includes, for example, a GPU, and processes data generated inside the automatic work machine 5 or received from the outside. Examples of the data to be processed by the data processing unit 206 include data indicating an operation record, data indicating environment information received from the server 7, and data indicating biological information received from the wearable device 3.

A camera 208 is a camera that photographs the outside world of the automatic work machine 5. A plurality of cameras 208 may be provided, and for example, a captured image of the left front and a captured image of the right front can be acquired. Moreover, the camera 208 includes a mechanism for adjusting the angle in the horizontal direction and a mechanism for adjusting the angle in the vertical direction. A notification unit 209 includes, for example, a lamp, an indicator, a speaker, or the like, and can notify the user 2 by sound or display. An operation unit 210 (control panel) includes a key such as a power button and a panel, and can receive an operation of the user 2.

A sensor group 211 includes various sensors related to the operation of the automatic work machine 5, and includes, for example, an orientation sensor, a speed sensor, an acceleration sensor, an obstacle detection sensor, a blade height sensor, a sensor that detects an area signal from an area wire, and a current sensor that detects the remaining amount of power. A GPS 212 receives a radio wave from a GPS satellite and acquires information indicating the current position (latitude, longitude) of the automatic work machine 5. A travel motor 213 drives a travel mechanism of the automatic work machine 5 such as wheels. A work motor 214 can drive a work device 215 and, for example, rotate the blade or adjust the height of the blade from the ground. A communication interface (I/F) 216 has a configuration corresponding to a communication medium such as an antenna, and enables communication with the outside. The communication I/F 216 can perform wireless communication such as Bluetooth or Wi-Fi, for example. A power supply I/F 217 is, for example, a charging terminal, and can receive power supply from a charging station by being connected with the charging terminal of the charging station. The communication control unit 203, the traveling control unit 204, the device control unit 205, and the data processing unit 206 perform each control processing on the basis of communication with each of the blocks from the storage unit 207 to the power supply I/F 217. Note that the configuration of the automatic work machine 5 is not limited to the block configuration illustrated in FIG. 2, and may appropriately include another block in accordance with function that can be implemented by the automatic work machine 5.

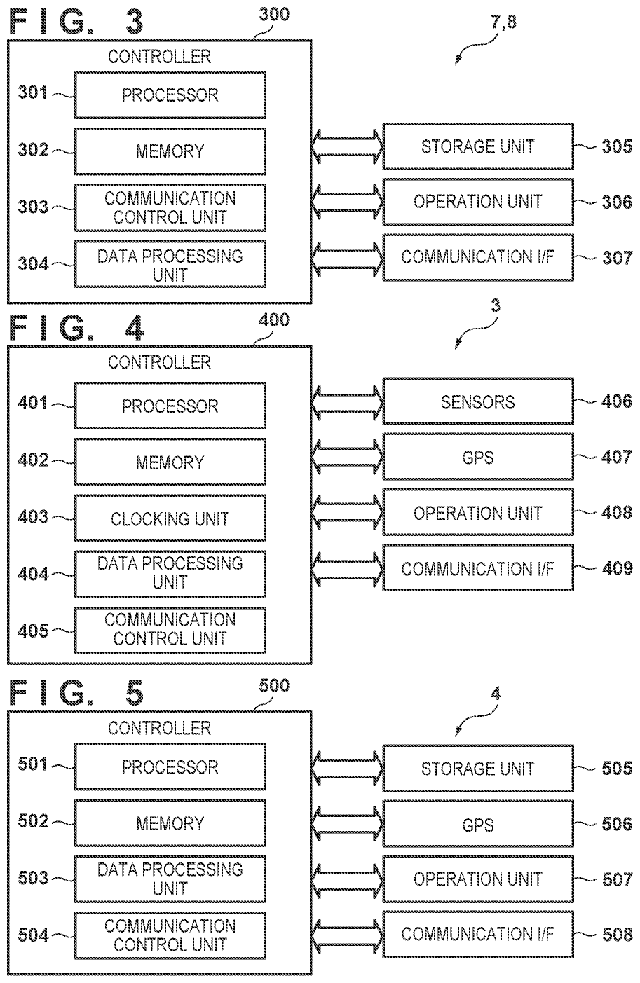

FIG. 3 is a diagram illustrating an example of the configurations of the server 7 and the server 8. The server 7 and the server 8 are configured as general information processing devices such as PCs. Hereinafter, the server 7 will be described as a representative example of the server 7 and the server 8 unless otherwise specified. A controller 300 is a control board for integrally controlling the server 7. The controller 300 includes a processor 301 such as a CPU, a memory 302 such as a ROM, an EEPROM, or a RAM, a communication control unit 303, and a data processing unit 304. The memory 302 stores a control program and data for controlling the operation of each unit of the server 7. Moreover, such programs and data may be stored in a storage unit 305 such as a hard disk configured outside the controller 300. The operation of the server 7 in the present embodiment is implemented by, for example, the processor 301 reading and executing a program stored in the memory 302. That is, a device including the controller 300 can be a computer in the present invention. The communication control unit 303 controls communication with the outside on the basis of a communication control program and data stored in the memory 302. For example, the communication control unit 303 of the server 7 controls communication with the wearable device 3 and the mobile terminal 4, communication with the automatic work machine 5, and communication with the server 8. The data processing unit 304 processes data generated inside the server 7 or received from the outside.

The storage unit 305 stores programs and data used in the present embodiment. For example, the storage unit 305 of the server 7 stores registered user information in the automatic work system 1, information on the automatic work machine 5 and the wearable device 3 corresponding to the user 2, and information regarding a service of the automatic work system 1 such as rewards. Moreover, a database based on big data may be configured in the storage unit 305. For example, the storage unit 305 of the server 7 may store the work result transmitted from the automatic work machine 5, the biological information from the wearable device 3, and the evaluation information for the work result in the work area 9 as big data, and the data processing unit 304 including the GPU may be configured to be able to analyze the tendency of the data. An operation unit 306 includes a hardware key and a panel, and can display various user interface screens to the user and accept user operations. A communication I/F 307 has a configuration corresponding to a communication medium, and enables communication with the outside.

Note that the configurations of the server 7 and the server 8 are not limited to the block configuration illustrated in FIG. 3, and can include other blocks as appropriate in accordance with functions that can be implemented by the server 7 and the server 8. Moreover, each of the server 7 and the server 8 may be configured as a single device or may be configured with a plurality of devices. Moreover, the server 7 and the server 8 may be configured as the same device. Moreover, some of the functions of the server 7 and the server 8 may be implemented by the automatic work machine 5.

FIG. 4 is a diagram illustrating an example of the configuration of the wearable device 3. A controller 400 is a control board for integrally controlling the wearable device 3. The controller 400 includes a processor 401 such as a CPU, a memory 402 such as a ROM, an EEPROM, or a RAM, a clocking unit 403, a data processing unit 404, and a communication control unit 405. The memory 402 stores a control program and data for controlling the operation of each unit of the wearable device 3. The operation of the wearable device 3 in the present embodiment is implemented by, for example, the processor 401 reading and executing a program stored in the memory 402. The clocking unit 403 has a time acquisition and clocking function related to acquisition of biological information. The data processing unit 404 processes a detection signal detected by a sensor group 406 or generates data to be transmitted as biological information to the outside, for example, on the basis of a program and data stored in the memory 402. The communication control unit 405 controls communication with the outside on the basis of a program and data stored in the memory 402. A communication I/F 409 has a configuration corresponding to a communication medium, and enables communication with the outside. For example, the wearable device 3 can be connected with the mobile terminal 4 in direct connection by a USB cable, or can be connected with the mobile terminal 4 and the automatic work machine 5 in short-range wireless communication such as Bluetooth.

The sensor group 406 is sensors for detecting biological information of the user 2 such as a heart rate, and is, for example, optical sensors that read a blood volume using an LED. A GPS 407 receives a radio wave from a GPS satellite and acquires information indicating the current position (latitude, longitude) of the wearable device 3. An operation unit 408 includes a power switch, a setting switch, and a panel, and can receive a user operation. Note that the configuration of the wearable device 3 is not limited to the block configuration illustrated in FIG. 4, and can suitably include another block in accordance with a function that can be implemented by the wearable device 3.

FIG. 5 is a diagram illustrating an example of the configuration of the mobile terminal 4. In the present embodiment, the mobile terminal 4 is, for example, a general information processing device such as a PC or a smartphone. A controller 500 is a control board for integrally controlling the mobile terminal 4. The controller 500 includes a processor 501 such as a CPU, a memory 502 such as a ROM, an EEPROM, or a RAM, a data processing unit 503, and a communication control unit 504. The memory 502 stores a control program and data for controlling the operation of each unit of the mobile terminal 4. The operation of the mobile terminal 4 in the present embodiment is implemented by, for example, the processor 501 reading and executing a program stored in the memory 502. The data processing unit 503 processes data generated inside the mobile terminal 4 or received from the outside on the basis of a program and data stored in the memory 502. The communication control unit 504 controls communication with the outside on the basis of a program and data stored in the memory 502.

A storage unit 505 stores a program and data for implementing the operation in the present embodiment. For example, the storage unit 505 stores an application for establishing data synchronization with the wearable device 3 and an application for receiving a notification from the automatic work machine 5. A GPS 506 receives a radio wave from a GPS satellite and acquires information indicating the current position (latitude, longitude) of the mobile terminal 4. An operation unit 507 includes a key such as a power switch and a panel, and can receive a user operation. A communication I/F 508 has a configuration corresponding to a communication medium, and enables communication with the outside. The communication I/F 508 can perform wireless communication such as Bluetooth or Wi-Fi, for example. Note that the configuration of the mobile terminal 4 is not limited to the block configuration illustrated in FIG. 5, and can suitably include another block in accordance with a function that can be implemented by the mobile terminal 4. For example, the mobile terminal 4 may have a function of acquiring biological information of the user 2.

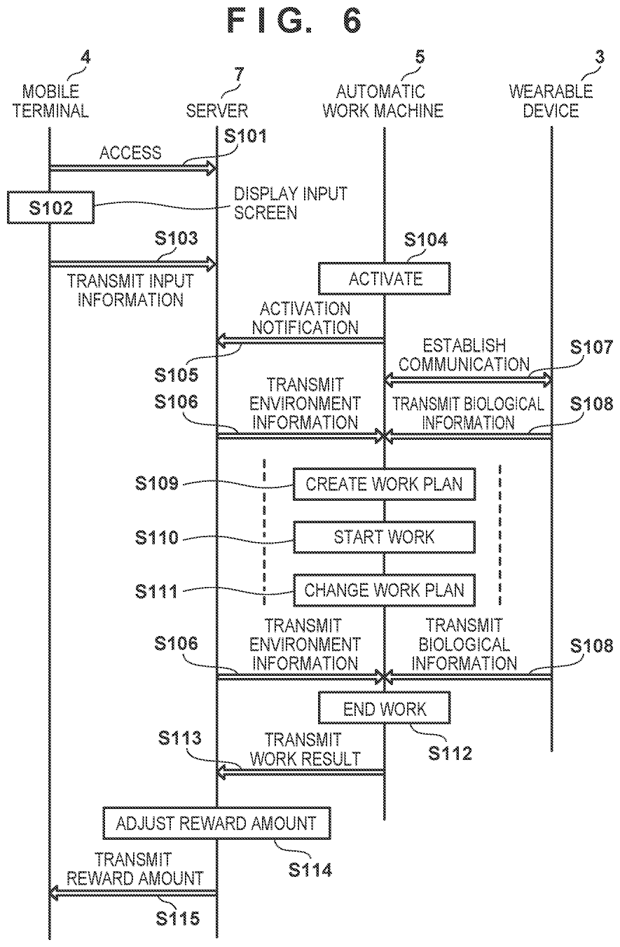

FIG. 6 is a sequence diagram for explaining the operation of the automatic work system 1. First, the mobile terminal 4 accesses a predetermined URL of the server 7 (S101). Although the description here will be given assuming that the mobile terminal 4 accesses the predetermined URL of the server 7, a device different from the mobile terminal 4 (e.g., a PC owned separately by the user 2) may access the predetermined URL of the server 7. By accessing the predetermined URL, a screen for registering user information in the automatic work system 1 is displayed on the mobile terminal 4 (S102). In addition to the information of the user 2, identification information (serial number, the number of machines, etc.) of the automatic work machine 5 held by the user 2 and identification information of the wearable device 3 are input on the screen for registering the user information. The information input in S102 is transmitted to and registered in the server 7 (S103). Note that the processes of S101 to S103 are not limited to be performed on a day when the user 2 performs work in the work area 9, and may be performed on or before the day.

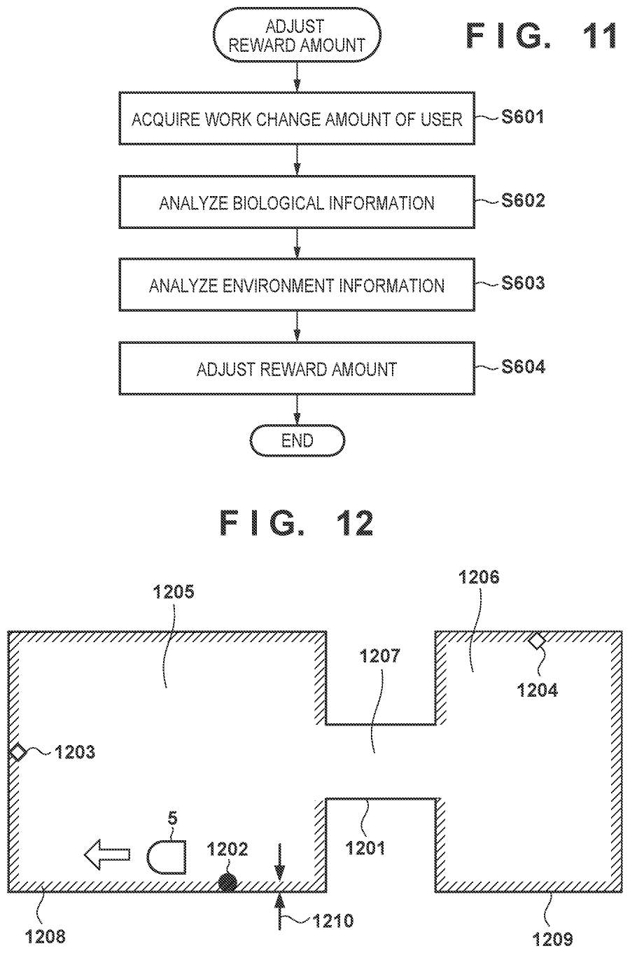

The user 2 lays an area wire and a charging station in order to partition the work area 9 for the mowing work. FIG. 12 is a diagram illustrating a state in which an area wire and a charging station are laid; In FIG. 12, an area 1205 and an area 1206 are partitioned as the work area 9 by laying an area wire 1201. The automatic work machine 5 is charged by being connected with a charging station 1202. The automatic work machine 5 can detect a signal from the area wire 1201. The automatic work machine 5 moves along the area wire 1201, turns inward when reaching a start point provided in each area, and starts work in the area. In the area, the automatic work machine 5 performs work in accordance with each travel mode such as random or zigzag. One start point is provided in each area. A start point 1203 is provided in the area 1205, and a start point 1204 is provided in the area 1206. The automatic work machine 5 moves between the areas 1205 and 1206 through a narrow path 1207.

When the user 2 activates the automatic work machine 5 (S104), notification information (activation notification) indicating the activation is transmitted from the automatic work machine 5 to the server 7 (S105). Then, a wearable device 3 present in a surrounding area is detected, and communication with the detected wearable device 3 is established (S107). After the communication with the wearable device 3 is established, the biological information of the user 2 is transmitted to the automatic work machine 5 at predetermined time intervals (S108). As a result, the automatic work machine 5 can monitor the biological information of the user 2.

On the other hand, when receiving an activation notification from the automatic work machine 5, the server 7 acquires, from the server 8, environment information of the vicinity of the position of the user 2. Note that the position information of the user 2 may be acquired on the basis of GPS information of the wearable device 3 or the mobile terminal 4. Moreover, the position information of the automatic work machine 5 may be used as position information of the user 2. The server 7 acquires the environment information at predetermined time intervals and transmits the environment information to the automatic work machine 5 (S106). As a result, the automatic work machine 5 can recognize the information on the environment in which the user 2 is present as required.

The automatic work machine 5 creates a work plan on the basis of the contents set in the setup wizard on the control panel (S109). The contents to be set are, for example, the length of the area wire, the area size, and the work mode. In the present embodiment, the work mode includes a cooperative work mode in which the automatic work machine 5 and the user 2 work in cooperation.

An example of the cooperative work mode will be described with reference to FIG. 12. Areas 1208 and 1209 indicated by hatching in FIG. 12 are worker responsible areas in which the user 2 performs work by himself/herself. That is, the user 2 performs the work by himself/herself instead of the automatic work machine 5 in edge areas where the automatic work machine 5 cannot sufficiently perform the work. In the case illustrated in FIG. 12, the automatic work machine 5 autonomously performs work inside the worker responsible areas 1208 and 1209. In a case where the cooperative work mode is set, the work plan of the user 2 and the work plan of the automatic work machine 5 are created on the basis of the worker responsible areas 1208 and 1209. Described in the present embodiment as an example of the cooperative work mode is a case where the automatic work machine 5 starts work from the start point 1203 and the user 2 performs mowing work in the worker responsible area 1208 in parallel with the work of the automatic work machine 5.

The automatic work machine 5 starts the work from a start point on the area wire 1201 (S110). The automatic work machine 5 monitors the biological information received from the wearable device 3 during work. In a case where the biological information satisfies a predetermined condition, the work plan created in S109 is changed (S111). The predetermined condition here is, for example, a condition with which it is determined that a change has occurred in the physical condition of the user 2, and is, for example, a condition that the heart rate is equal to or greater than a threshold. In this case, the work plan is changed to reduce the load on the user 2. Even in a case where the biological information does not satisfy the predetermined condition, the work plan created in S109 may be changed in a case where the environment information satisfies a predetermined condition. The predetermined condition of the environment information is, for example, a condition that is determined to be an environmental factor that lowers the work efficiency of the user 2, and is, for example, an increase in temperature. In this case, the work plan is changed to reduce the load on the user 2.

At the end timing of the work, the automatic work machine 5 returns to the charging station 1202 and ends the work (S112). The work end timing is, for example, a timing at which the work defined in the work plan is completed, or a timing at which the work ending time comes. Then, the automatic work machine 5 transmits the work result to the server 7 (S113). The work result to be transmitted includes, for example, information on work completion, transition of biological information of the user 2 and environment information, and change contents (presence or absence of change, etc.) of the work plan.

The server 7 adjusts the reward amount to be paid to the user 2 on the basis of the work result received in S113. For example, the reward amount is increased in a case where it is estimated from the transition of the heart rate and the environment information that the worker has been working while feeling stress in an environment with an extremely high temperature. As described above, in the present embodiment, it becomes possible to increase motivation of the user 2 for the work. The server 7 transmits information on the reward amount adjusted in S114 to the mobile terminal 4 (S115).

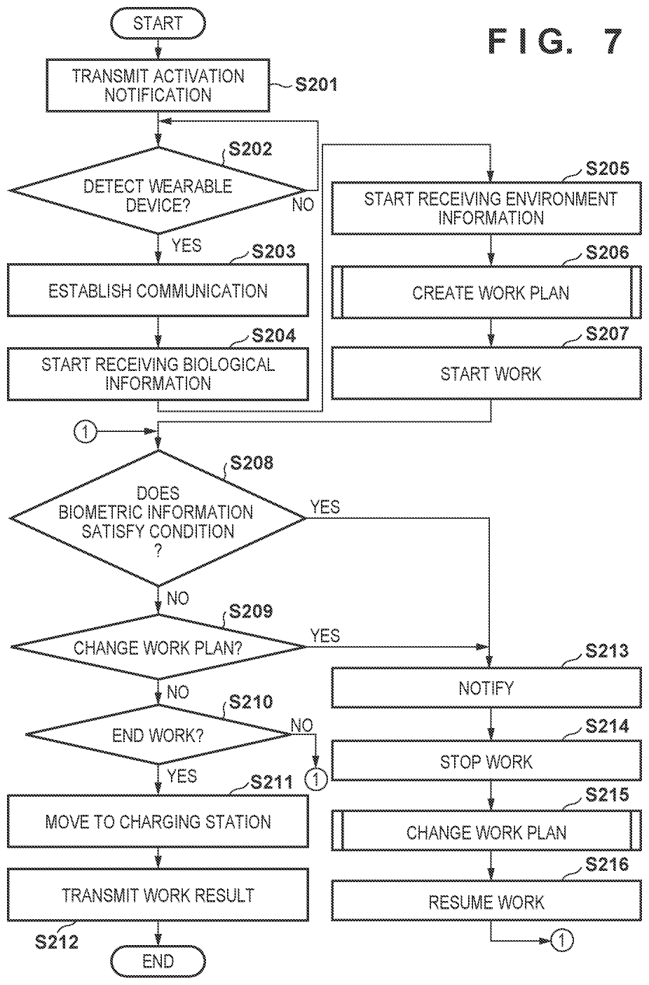

FIG. 7 is a flowchart illustrating processing of the automatic work machine 5. The processing in FIG. 7 is started when the processing is activated in S104 of FIG. 6. In S201, the controller 200 of the automatic work machine 5 transmits notification information indicating the activation to the server 7. The notification information indicating the activation may include position information of the automatic work machine 5. In S202, the controller 200 determines whether a wearable device 3 in a surrounding area has been detected or not. Here, the process of S202 is repeated until it is determined that a wearable device 3 has been detected, and the processing proceeds to S203 in a case where it is determined that a wearable device 3 has been detected. S201 corresponds to S105 in FIG. 6.

In S203, the controller 200 establishes communication with the detected wearable device 3, and in S204, starts receiving the biological information of the user 2 detected by the wearable device 3. Note that the controller 200 may acquire the position information of the wearable device 3 and transmit the position information to the server 7 when communication is established. In S202 and S203, detection of the mobile terminal 4 and acquisition of the position information may be performed instead of or together with the wearable device 3. In S205, the controller 200 starts receiving the environment information from the server 7. In S206, the controller 200 creates a work plan. S203, S204, S205, and S206 correspond to S107, S108, S106, and S109 in FIG. 6.

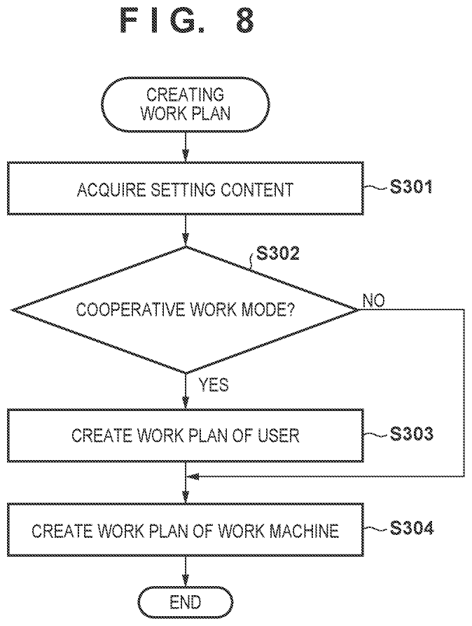

FIG. 8 is a flowchart illustrating processing of creating a work plan in S206. In S301, the controller 200 acquires the setting contents in the setup wizard displayed on the operation unit 210. The setting contents acquired here include the length of the area wire 1201, the area size of the area, the width of the narrow path 1207, the position of the charging station 1202, and the positions of the start points 1203 and 1204. Moreover, the setting contents include information (width 1210, length, etc.) on the work mode and the worker responsible area. Moreover, the number of automatic work machines 5 may be set, or a plurality of automatic work machines 5 may be set in the work area 9. In this case, for example, the number of automatic work machines and the identification information of the automatic work machine 5 may be set for each area, such as one in the area 1205 and one in the area 1206.

In S302, the controller 200 determines whether the set work mode is the cooperative work mode or not. In a case where it is determined that the work mode is not the cooperative work mode, the processing proceeds to S304, a work plan for the automatic work machine 5 to autonomously perform work is created, and the processing in FIG. 8 ends. On the other hand, in a case where it is determined that the work mode is the cooperative work mode, the controller 200 creates a work plan of the user 2 in S303.

The work plan of the user 2 is information on the worker responsible areas 1208 and 1209 where the user 2 performs work. For example, the width 1210 and the length of the worker responsible area are set as such information. For the work plan of the user 2, the setting contents input from the user 2 in the setup wizard may be used, or the setting contents input from the user 2 may be changed on the basis of the biological information of the user 2. For example, according to the biological information transmitted from the wearable device 3 in S108 after the communication is established in S107, the setting contents input by the user 2 may be changed so as to reduce the work amount of the user 2 when the heart rate is higher than a predetermined value. At that time, a change instruction from the user 2 may be accepted on the operation unit 210. Instead of or in addition to the biological information, a change may be made on the basis of the environment information transmitted from the server 7 in S106.

After S303, the controller 200 creates, in S304, a work plan of the automatic work machine 5 on the basis of the work plan of the worker created in S303. For example, a work plan for the automatic work machine 5 to autonomously perform work is created on the basis of the area 1205 excluding the worker responsible area 1208. After S304, the processing in FIG. 8 ends.

After the work plan is created in S206, the controller 200 leaves the charging station 1202 and moves along the area wire 1201 in S207. Then, when the automatic work machine 5 reaches the start point 1203, the controller 200 controls the automatic work machine 5 to perform turning operation, and starts the work. Note that the user 2 starts the work in the worker responsible area 1208 by himself/herself. S207 corresponds to S110 in FIG. 6.

In S208, the controller 200 determines whether the biological information received from the wearable device 3 in S108 satisfies a condition or not. The condition here is, for example, that the heart rate of the user 2 has increased to a value equal to or greater than a threshold. In a case where it is determined that the biological information satisfies the condition, the controller 200 notifies the user 2 by sound or display in S213, and performs control to stop the work of the work device 215 in S214.

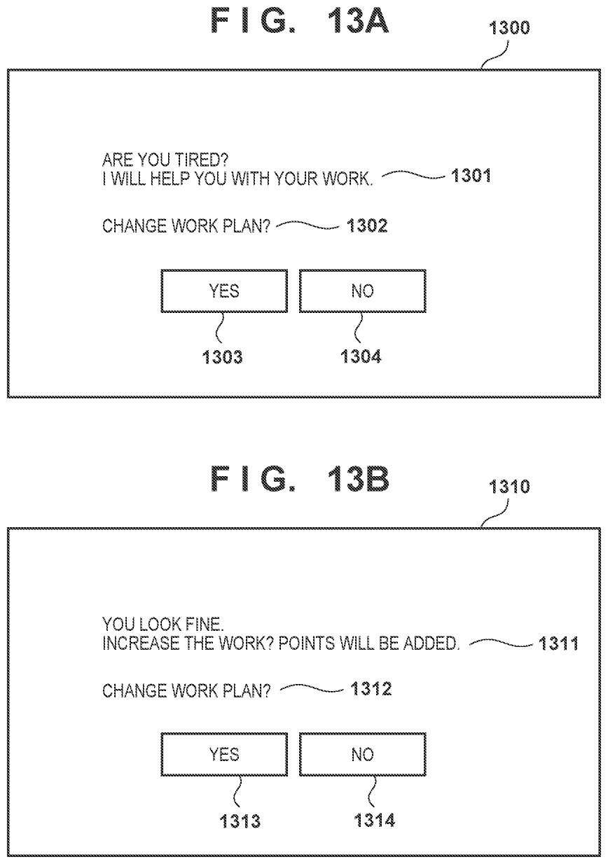

In a case where the work of the work device 215 is stopped in S214, a screen illustrated in FIG. 13A is displayed on the operation unit 210 in S215. For example, in a case where it is determined in S208 that the heart rate has increased to a value equal to or greater than the threshold, a screen 1300 illustrated in FIG. 13A is displayed. On the screen 1300, a message of “Are you tired? I will help you with your work.” is displayed as a message 1301. That is, in the present embodiment, the physical condition of the user 2 is monitored during work, and a message such as the message 1301 expressing appreciation to the user 2 is displayed in a case where the tendency of fatigue or the like can be recognized. The message 1301 is not limited to the message illustrated in FIG. 13A, and may indicate encouragement or the like such as “Please do your best”. Moreover, the type of message may be changed in accordance with the condition of the determination criterion. For example, an encouraging message may be displayed in a case where a threshold A is exceeded, and a message to propose work cooperation such as “Do you want help with the work?” may be displayed in a case where a threshold B higher than the threshold A is exceeded. Moreover, such setting of a plurality of thresholds may be decided on the basis of the acquisition result of the biological information at that time at the stage of creating the work plan in S109. Moreover, the above message may be output by voice.

The screen 1300 further displays a message proposing to change the work plan as a message 1302. The message 1302 may be displayed, for example, in a case where a message suggesting cooperation in work is displayed in the message 1301. When the message 1302 is displayed, the controller 200 waits for pressing of a button 1303 for accepting an instruction to change the work plan or a button 1304 for accepting an instruction not to change the work plan.

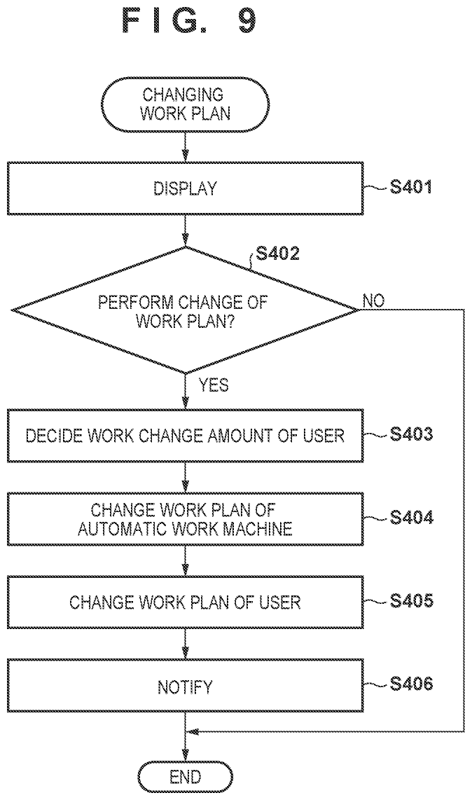

FIG. 9 is a flowchart illustrating processing of changing the work plan in S215. In S401, the controller 200 displays the screen 1300 in FIG. 13A on the operation unit 210. Then, in S402, the controller 200 determines whether to change the work plan or not on the basis of the pressing of the button 1303 or 1304. In a case where it is determined that the work plan is not to be changed, the processing in FIG. 9 ends, and the controller 200 performs control to resume the work of the automatic work machine 5 in S216. Thereafter, the processes from S208 are repeated.

On the other hand, in a case where it is determined to change the work plan, the controller 200 decides the work change amount of the user 2 in S403. For example, if the worker responsible area after the point where the work is currently stopped is to be changed to the target of the autonomous work by the automatic work machine 5, the worker responsible area after the current point is decided as the work decrease of the user 2. Moreover, for example, if a point ahead of a point where the work is currently stopped by a predetermined distance (e.g., 4 m) is changed to a target of the autonomous work by the automatic work machine 5, the worker responsible area after the point is decided as the work decrease of the user 2. Moreover, the predetermined distance may be decided on the basis of biological information and environment information at that time. For example, the predetermined distance may be decided to be decreased, as the heart rate is higher or the temperature is higher. Moreover, the predetermined distance may be decided on the basis of the work content of the user 2 up to the current time point. For example, if it is determined from the photographing data of the camera 208 that the movement amount of the user 2 is equal to or less than a predetermined value, it may be determined that the work pace of the user 2 is lowering, and the predetermined distance may be decided to be shortened. It is possible with such a configuration to change the work plan in consideration of the state of the user 2 more accurately.

In S404, the controller 200 changes the work plan of the automatic work machine 5. For example, the controller 200 changes the work plan of the automatic work machine 5 so as to compensate for the work decrease of the user 2 decided in S403. For example, the work plan is changed so that the worker responsible area after the current point becomes a target of autonomous work by the automatic work machine 5. The controller 200 changes the work plan by changing parameters for the automatic work machine 5 to autonomously perform work. Such parameters include, for example, a work area size and a work time. Moreover, the controller 200 may change the work plan of the automatic work machine 5 on the basis of a work schedule preset for the work area 9. For example, in a case where it is estimated that the work cannot be ended within a preset work ending time if the work plan of the automatic work machine 5 is changed to compensate for the work decrease of the user 2, the work target may be narrowed down to a partial work area (only the area 1205, etc.). In this case, the controller 200 changes the work plan of the automatic work machine 5 so as to work only in the narrowed target area.

In S405, the controller 200 changes the work plan of the user 2. For example, the controller 200 changes the work plan of the user 2 in accordance with the work decrease of the user 2 decided in S403. For example, the work plan is changed so that a point that is 4 m ahead of the current point is set as a work target of the user 2. In S406, the controller 200 notifies the user 2 of the contents of the work plan changed in S405. For example, a message of “Please finish your work in 4 m. I will help you with the rest.” may be displayed on the operation unit 210. Moreover, the message may be output not by display but by voice. Moreover, the above message may be transmitted to the mobile terminal 4. After S406, the processing in FIG. 9 ends, and the controller 200 performs control to resume the work of the automatic work machine 5 in S216. Thereafter, the processes from S208 are repeated. S215 corresponds to S111 in FIG. 6.

In a case where it is determined in S208 that the biological information received from the wearable device 3 does not satisfy the condition, the controller 200, in S209, determines whether to change the work plan or not on the basis of the environment information received from the server 7 in S106. For example, in a case where the temperature is equal to or higher than the threshold, it may be determined to change the work plan. For example, in a case where the temperature is high even if there is no large fluctuation in the heart rate, the work plan can be changed by the processes in and after S213. It is possible with such a configuration to propose a change of the work plan before the user 2 starts feeling fatigue. In a case where the processing proceeds from S209 to S213, for example, the work decrease of the user 2 may be set for each predetermined increase in temperature in S403. In a case where it is determined in S209 that the work plan is not to be changed, the processing proceeds to S210.

In S210, the controller 200 determines whether it is a timing to end the work of the automatic work machine 5 or not. For example, the controller 200 determines whether it is a timing to end the work of the automatic work machine 5 on the basis of a timing at which the work defined in the work plan is ended or a timing at which the work ending time comes. S210 corresponds to S112 in FIG. 6.

When it is determined in S210 that it is the timing to end the work of the automatic work machine 5, the controller 200 causes the automatic work machine 5 to move to and connect to the charging station 1202 in S211. Then, in S212, the controller 200 transmits the work result to the server 7. The work result includes, for example, information on work completion, transition of biological information of the user 2 and environment information, and change contents (presence or absence of change, etc.) of the work plan. Then, the processing in FIG. 7 ends. S212 corresponds to S113 in FIG. 6.

A case where the work amount of the user 2 is reduced has been described above. However, the operation of the present embodiment can also be applied to a case where the work amount of the user 2 is increased. For example, in a case where it is determined in S208 that the biological information received from the wearable device 3 does not satisfy the condition and it is determined in S209 that the work plan is to be changed on the basis of the environment information, a screen 1310 in FIG. 13B may be displayed in S401. On the screen 1310, a message 1311 such as “You look fine. Increase the work? Points will be added.” is displayed. That is, for example, in a case where it is determined that there is no large fluctuation in the heart rate even though the temperature is equal to or higher than a threshold, it may be determined that the physical condition is good, and a message suggesting an increase in the work amount may be displayed. At that time, a message to add points may be displayed. Here, the points may be, for example, money such as a salary, or may be exchangeable into money. The screen 1310 further displays a message proposing to change the work plan as a message 1312. When the message 1312 is displayed, the controller 200 waits for pressing of a button 1313 for accepting an instruction to change the work plan or a button 1314 for accepting an instruction not to change the work plan.

In a case where it is determined in S402 that the work plan is to be changed by pressing of the button 1313, when an area further advanced by a predetermined distance (e.g., 10 m) from the scheduled work end point of the user 2 is changed to the work area of the user 2, the controller 200 decides that amount as the work increase of the user 2 in S403, for example. The predetermined distance may be decided on the basis of biological information and environment information at that time. For example, the predetermined distance may be decided to be longer as the heart rate fluctuation rate is smaller. It is possible with such a configuration to change the work plan in consideration of the state of the user 2 more accurately.

In S404, the controller 200 changes the work plan of the automatic work machine 5 so as not to overlap with the work increase of the user 2 decided in S403. In S405, the controller 200 changes the work plan of the user 2 in accordance with the work increase of the user 2 decided in S403. In S406, the controller 200 notifies the user 2 of the contents of the work plan changed in S405. At that time, for example, an encouraging message such as “Please do your best.” may be displayed. Moreover, the message may be output not by display but by voice. Moreover, the above message may be transmitted to the mobile terminal 4. After S406, the processing in FIG. 9 ends, and the controller 200 performs control to resume the work of the automatic work machine 5 in S216. Thereafter, the processes from S208 are repeated.

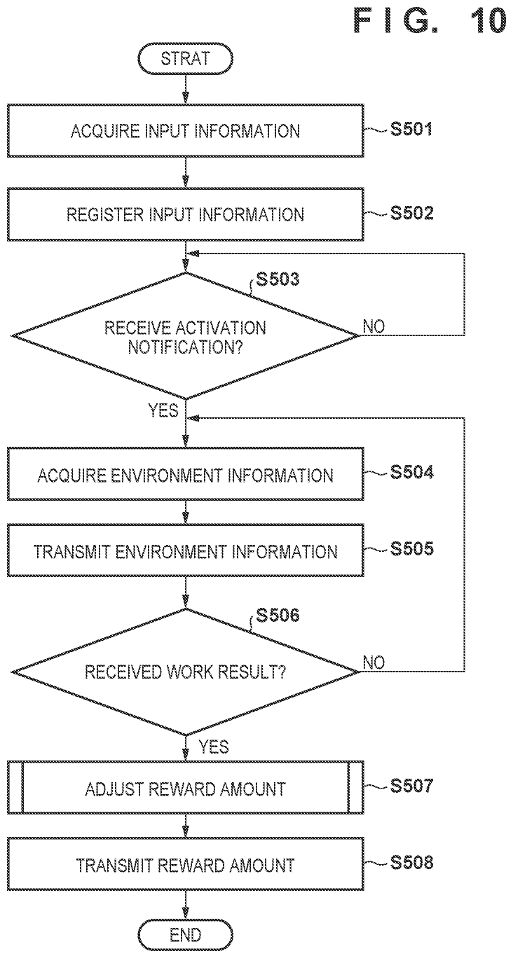

FIG. 10 is a flowchart illustrating processing by the server 7. In S501, the controller 300 of the server 7 acquires the input information transmitted from the mobile terminal 4 in S103. Then, in S502, the controller 300 registers the input information acquired in S501 in the storage unit 305. The storage unit 305 stores identification information of the user 2, identification information (serial number, the number of machines, etc.) of the automatic work machine 5, and identification information of the wearable device 3 in association with each other.

In S503, the controller 300 monitors reception of notification information indicating activation from the automatic work machine 5. In a case where the notification information indicating activation is received, the controller 300 acquires the environment information from the server 8 in S504. S503 corresponds to S105 in FIG. 6. The environment information may be acquired on the basis of any of the position information of the automatic work machine 5, the position information of the wearable device 3, and the position information of the mobile terminal 4. In S505, the controller 300 transmits the environment information received in S504 to the automatic work machine 5. S505 corresponds to S106 in FIG. 6.

In S506, the controller 300 determines whether a work result has been received from the automatic work machine 5 or not. In a case where it is determined that a work result has not been received from the automatic work machine 5, that is, in a case where the automatic work machine 5 is working, the processes from S504 are repeated. That is, in the present embodiment, environment information on the environment where the user 2 is present is transmitted to the automatic work machine 5 as required during work of the automatic work machine 5. With such a configuration, the automatic work machine 5 can recognize the environment information on the environment where the user 2 is present as required.

In a case where it is determined in S506 that a work result from the automatic work machine 5 has been received, the controller 300 adjusts the reward amount of the user 2 on the basis of the received work result in S507. S507 corresponds to S114 in FIG. 6.

FIG. 11 is a flowchart illustrating processing of adjusting the reward amount in S507. In S601, the controller 300 acquires the work change amount of the user 2 from the work result received from the automatic work machine 5. The work change amount is the work change amount decided in S403. Here, the work change amount includes a case where the work amount is not changed, in addition to a case where the work is decreased and a case where the work is increased. In S602, the controller 300 extracts and analyzes the biological information from the work result received from the automatic work machine 5. For example, the controller 300 analyzes the transition of the heart rate during the work of the user 2. In S603, the controller 300 extracts and analyzes the environment information from the work result. For example, the controller 300 analyzes the transition of the temperature during the work of the user 2.

In S604, the controller 300 adjusts the reward amount on the basis of the work change amount acquired in S601 and the analysis results in S602 and S603. For example, the reward amount may be increased in a case where the work change amount indicates an increase in the work amount. Even in a case where the work amount is not changed, the reward amount may be increased in a case where the analysis result of the biological information indicates, for example, a tendency of an increase in the heart rate. Moreover, the reward amount may be increased in a case where the analysis result of the environment information indicates a factor that lowers the work efficiency, such as an increase in the temperature or rainfall, for example. In a case where the work plan is changed so as to work only in a narrowed target area such as only the area 1205 in S404 and S405, the reward amount may be decreased. Moreover, evaluation information of the work area 9 where the work has been performed may be acquired, and the reward amount may be increased or decreased on the basis of the evaluation information. Such evaluation information may be, for example, acquired on a homepage provided by the server 7 and stored in the storage unit 305. Moreover, the above cases may be combined. After S604, the processing in FIG. 11 ends, and the controller 300 transmits, in S508, the reward amount adjusted in S507 to the mobile terminal 4. S508 corresponds to S115 in FIG. 6. As described above, in the present embodiment, the reward amount is adjusted so that motivation of the user 2 for the work can be maintained or improved.

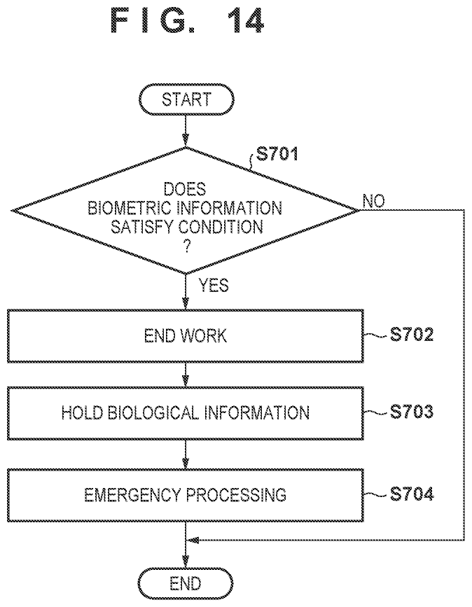

Hereinafter, emergency processing performed in a case where the biological information of the user 2 indicates an abnormal state or the like will be described. FIG. 14 is a flowchart illustrating emergency processing executed by the automatic work machine 5. The processing in FIG. 14 is performed as interrupt processing during monitoring of the biological information transmitted from the wearable device 3 in S108.

In S701, the controller 200 determines whether the biological information satisfies a condition for executing the processing in FIG. 14 or not. The condition here is, for example, that the biological information is out of a range recognized as the normal health condition. In a case where it is determined in S701 that the condition is not satisfied, the processing in FIG. 14 ends, and the processing in FIG. 7 is executed. On the other hand, in a case where it is determined in S701 that the condition is satisfied, the controller 200 forcibly terminates the work performed by the work device 215 in S702. In S703, the controller 200 holds the currently received biological information of the user 2 in a non-volatile storage area such as the storage unit 207. Then, in S704, the controller 200 executes emergency processing.

For example, as the emergency processing, the controller 200 may perform control to move to the vicinity of the user 2 and cause the notification unit 209 to make a warning sound or the like. Moreover, the controller 200 may switch the user interface to voice guidance to check the state of the user 2. At that time, in a case where there is no response from the user 2, emergency notification to the server 7 or notification to a family member or the like of the user 2 may be performed. The contact to the family member or the like of the user 2, the type of emergency processing, and the like may be input on the input screen in S102. As described above, in the present embodiment, in a case where the monitoring result of the biological information indicates an abnormal state, the work can be forcibly terminated, and emergency processing for preferentially checking the state of the user 2 can be performed.

As described above, it is possible with the present embodiment to monitor the state of the user 2 and appropriately change the work plans of the automatic work machine 5 and the user 2 in accordance with the monitoring result. As a result, it is possible to balance the load of the user 2 and the work efficiency.

### Summary of Embodiments

The work system of the above embodiment includes an acquisition unit (S108) that acquires biological information of a user, a creation unit (S109) that creates a work plan in a work area, a control unit (controller 200) that controls a work device for performing work in the work area on the basis of the work plan created by the creation unit, and a change unit (S111, S404) that changes the work plan created by the creation unit on the basis of the biological information acquired by the acquisition unit. In a case where the work plan created by the creation unit is changed by the change unit, the control unit controls the work device on the basis of the changed work plan (S216).

It is possible with such a configuration to appropriately change the work plan of the automatic work machine 5 in accordance with the state of the user 2.

Moreover, the creation unit creates the work plan on the basis of the biological information acquired by the acquisition unit. It is possible with such a configuration to create a work plan in accordance with the state of the user 2.

Moreover, the acquisition unit repeatedly acquires the biological information while the work device is controlled by the control unit. It is possible with such a configuration to create a work plan on the basis of a change in the state of the user 2.

Moreover, the biological information includes at least one of a heart rate, a pulse, a blood pressure, a body temperature, or a respiratory amount. It is possible with such a configuration to create a work plan on the basis of the heart rate of the user 2, for example.

Moreover, the change unit changes the work plan by changing at least one of a work area size or a work time. It is possible with such a configuration to change the created work plan by changing the work area size, for example.

The change unit further changes the work plan of the user (S405). The change unit changes the work plan created by the creation unit so as to compensate for a change in the work plan of the user. It is possible with such a configuration to perform the work for which the user 2 is in charge, for example, on behalf of the user 2.

Moreover, a second acquisition unit (S106) that acquires environment information of the work area is further provided, and the change unit changes the work plan created by the creation unit on the basis of the environment information acquired by the second acquisition unit. Moreover, the environment information includes weather information. It is possible with such a configuration to create a work plan in consideration of the load on the user more accurately.

Moreover, a third acquisition unit (S105) that acquires position information of the user is further provided, and the second acquisition unit acquires the environment information on the basis of the position information acquired by the third acquisition unit. It is possible with such a configuration to acquire environment information corresponding to the position of the user 2.

Moreover, the change unit changes the work plan created by the creation unit on the basis of the work content of the user. It is possible with such a configuration to change the work plan on the basis of the work pace of the user, for example.

In a case where the biological information acquired by the acquisition unit satisfies a predetermined condition, the control unit causes the work device to end the work in the work area regardless of the work plan created by the creation unit (S701 to S704). With such a configuration, for example, it is possible to forcibly terminate the work in a case where an abnormality is observed in the physical condition of the user 2.

Moreover, a decision unit (S114) that decides a reward for the user after completion of work in the work area is further provided. The decision unit decides a reward for the user on the basis of evaluation information for work in the work area. The decision unit decides a reward for the user on the basis of the biological information acquired by the acquisition unit. It is possible with such a configuration to decide the reward on the basis of, for example, the evaluation information for the work result acquired on the homepage or the transition of the heart rate of the user 2 during work.

Moreover, the creation unit, the control unit, the acquisition unit, and the change unit are characterized by being configured as a work machine (automatic work machine 5) provided with the work device. It is possible with such a configuration to implement an automatic work machine that appropriately changes the work plan of the automatic work machine 5 in accordance with the state of the user 2.

The invention is not limited to the foregoing embodiments, and various variations/changes are possible within the spirit of the invention.

What is claimed is:

1. A work system comprising:
an acquisition unit configured to acquire biological information of a user;
a creation unit configured to create a work plan in a work area;
a control unit configured to control a work device for performing work in the work area on the basis of the work plan created by the creation unit; and
a change unit configured to change the work plan created by the creation unit on the basis of the biological information acquired by the acquisition unit,
wherein the control unit controls the work device on the basis of the changed work plan in a case where the work plan created by the creation unit is changed by the change unit.

2. The work system according to claim 1, wherein the creation unit creates the work plan on the basis of the biological information acquired by the acquisition unit.

3. The work system according to claim 1, wherein the acquisition unit repeatedly acquires the biological information while the work device is controlled by the control unit.

4. The work system according to claim 1, wherein the biological information includes at least one of a heart rate, a pulse, a blood pressure, a body temperature, or a respiratory amount.

5. The work system according to claim 1, wherein the change unit changes the work plan by changing at least one of a work area size or a work time.

6. The work system according to claim 1, wherein the change unit further changes a work plan of the user.

7. The work system according to claim 6, wherein the change unit changes the work plan created by the creation unit so as to compensate for a change in the work plan of the user.

8. The work system according to claim 1, further comprising a second acquisition unit configured to acquire environment information of the work area,
wherein the change unit changes the work plan created by the creation unit on the basis of the environment information acquired by the second acquisition unit.

9. The work system according to claim 8, wherein the environment information includes weather information.

10. The work system according to claim 8, further comprising a third acquisition unit configured to acquire position information of the user,
wherein the second acquisition unit acquires the environment information on the basis of the position information acquired by the third acquisition unit.

11. The work system according to claim 1, wherein the change unit changes the work plan created by the creation unit on the basis of work content of the user.

12. The work system according to claim 1, wherein the control unit causes the work device to end work in the work area regardless of the work plan created by the creation unit in a case where the biological information acquired by the acquisition unit satisfies a predetermined condition.

13. The work system according to claim 1, further comprising a decision unit configured to decide a reward for the user after work in the work area ends.

14. The work system according to claim 13, wherein the decision unit decides a reward for the user on the basis of evaluation information for work in the work area.

15. The work system according to claim 13, wherein the decision unit decides a reward for the user on the basis of the biological information acquired by the acquisition unit.

16. The work system according to claim 1, wherein the creation unit, the control unit, the acquisition unit, and the change unit are configured in a work machine provided with the work device.

17. A control method executed in a work system, the control method comprising:
acquiring biological information of a user;
creating a work plan in a work area;
controlling a work device for performing work in the work area on the basis of the created work plan; and
changing the created work plan on the basis of the acquired biological information,
wherein the work device is controlled on the basis of the changed work plan in a case where the created work plan is changed.

18. A non-transitory computer-readable storage medium storing a program for causing a computer to function to:
acquire biological information of a user;
create a work plan in a work area;
control a work device for performing work in the work area on the basis of the created work plan; and
change the created work plan on the basis of the acquired biological information,
wherein the work device is controlled on the basis of the changed work plan in a case where the created work plan is changed.
